(19) United States
(12) Patent Application Publication
(10) Pub. No.: US 2010/0075806 A1
(43) Pub. Date: Mar. 25, 2010

(54) BIORHYTHM FEEDBACK SYSTEM AND METHOD

(76) Inventor: Michael Montgomery, North Salem, NY (US)

Correspondence Address: BOND, SCHOENECK & KING, PLLC, ONE LINCOLN CENTER, SYRACUSE, NY, 13202-1355, US

(21) Appl. No.: 12/410,271

(22) Filed: Mar. 24, 2009

Related U.S. Application Data
(60) Provisional application No. 61/070,575, filed on Mar. 24, 2008.
(60) Provisional application No. 61/134,411, filed on Jul. 10, 2008.
(60) Provisional application No. 61/203,833, filed on Dec. 30, 2008.

(51) Int. Cl. A63B 71/00; G09B 19/00; A61F 5/58

(52) U.S. Cl. 482/8; 434/258; 600/23

(57) ABSTRACT

A method and system for evaluating movement of a user and providing biofeedback including setting a reference point for movement by the user, whereby the reference point corresponds to a reference rhythm, providing the reference rhythm to the user to assist the user in maintaining the reference point, sensing the user's movement, comparing the user's movement to the reference point movement, and alerting the user that the user's movement is away from the reference point by modifying the reference rhythm to a modified rhythm. The user's movement is corrected after receiving the alert.

### RELATED APPLICATION

The present application claims priority to the U.S. provisional patent applications: (i) U.S. provisional patent application No. 61/070,575, filed Mar. 24, 2008; (ii) U.S. provisional patent application No. 61/134,411, filed on Jul. 10, 2008; and (iii) U.S. provisional patent application No. 61/203,833, filed on Dec. 30, 2008; all of the foregoing patent-related document(s) are hereby incorporated by reference herein in their respective entirety(ies).

### TECHNICAL FIELD

Embodiments of the present invention relate generally to biofeedback method and system and more specifically to a method and system for correcting movement in a person using biofeedback.

### BACKGROUND OF THE INVENTION

Rhythmic Auditory Stimulation is a method whereby audible rhythm is used to improve the sensorimotor coordination of people with neurological damage. It has often been delivered by setting a periodic sound, such as a metronome, to prompt people to move in synchrony with its' tempo.

Conditions requiring rehabilitation have always posed significant challenges to medical practitioners. One cause related to the challenge of treating patients is that medical practitioners have incomplete information with which to assess patients. They must use tools, such as diagnostic devices and tedious procedures such as standardized questioning, when assessing patients. These tools yield incomplete views of medical conditions, which are often affected by a number of factors such as lifestyle, diet, exercise, and biomechanics, among many other factors. Additionally, medical practitioners often have very limited time to assess patients, which further constrains their ability to gain thorough assessments of medical conditions.

Medical practitioners are also increasingly expected to base their clinical decisions on the best scientific evidence available, which can usually be found in academic journals and online databases. One standardized questioning approach used by practitioners is often informed by Evidence-Based Practice (EBP), which attempts to make decisions by identifying such evidence that there may be for a practice, and rating it according to how scientifically sound it may be. Its goal is to eliminate unsound or excessively risky treatments in favor of those that have better outcomes.

Usage of Electronic Medical Records (EMRs), which refers to the electronic storage of medical data, has risen sharply in recent years. EMR systems have been used to improve the efficiency and delivery of care in various health settings. An example of an existing EMR application used in medical rehabilitation is billing and documentation software, which records patients' medical information as well as information relevant to billing, transcription, and reimbursement, among other functions.

Health-related monitoring devices have proliferated in recent years. Such devices have enabled medical practitioners and caregivers to monitor patients in various settings with great precision and accuracy. For example, EKG machines have often been used to closely monitor patients recovering from cardiac arrest. Similarly, glucose monitors have commonly been used to improve the treatment of diabetes. Wireless communication greatly expands the potential of medical monitoring technologies to be used at patients' homes with great frequency.

Biofeedback is a known technique that conveys information to a person (or animal) that represents involuntary or unconscious bodily functions. For example, a person's heartbeat is measured and information representing the heartbeat, such as a beating sound, is conveyed to the person substantially at the same time as the heart is beating (e.g., in real-time). The information (e.g., the audible beats representing the person's heartbeat) enables the person to become aware of the function, thereby enabling the person to gain control and to affect the otherwise unconscious or involuntary function. Thus, a person who can hear his heartbeat amplified (or see his heartbeat represented in a graph) can use that information to gain control of his heartbeat and to increase or decrease his heart rate. Other physiological functions can similarly be affected via biofeedback, such as blood pressure, muscle tension, skin temperature or the like.

Biofeedback has been used as a medical treatment for various disorders and conditions. For example, biofeedback medical treatment is useful for upper extremity disorders, back and lower extremity injuries, chronic pain disorders and traumatic brain injuries. Treatments typically involve improving of a patient's awareness of a particular condition to enable the patient to affect physical responses and symptoms. Therefore, treatment of various disorders via biofeedback is one method for combating the escalating costs associated with healthcare treatment.

### SUMMARY OF THE INVENTION

It is a primary object of an embodiment of the present invention to provide a method for evaluating movement of a user and providing biofeedback including setting a reference point for movement by the user, whereby the reference point corresponds to a reference rhythm, providing the reference rhythm to the user to assist the user in maintaining the reference point, sensing the user's movement, comparing the user's movement to the reference point movement, and alerting the user that the user's movement is away from the reference point by modifying the reference rhythm to a modified rhythm. It is preferable that the user's movement is corrected after receiving the alert.

According to another aspect of the method, the reference rhythm is modified by increasing or decreasing the amplitude of the rhythm.

According to a further aspect of the method, the reference rhythm includes one or more patterns of movements. The pattern of movements can include music and/or beat signals. It is preferable that the music and beat signals are in synchronization. The music can include rock, pop, classical, jazz, hip hop, blues, alternative rock, rap music or a combination thereof. The beat signals can include sounds from a musical instrument or a metronome or a combination thereof. Examples of musical instrument include drums, symbols, a wind instrument, a string instrument, a piano, a harpsichord, an organ, or a combination thereof.

According to yet another aspect of the method, the amplitude is increased by increasing the amplitude of the music, the beat signals or both and the amplitude is decreased by decreasing the amplitude of the music, the beat signals or both. The amplitude of the rhythm can be increased or decreased at specific parts of the pattern.

According to a further aspect of the method, the rhythm can be further modified by increasing or decreasing the tempo, timbre, frequency, pitch, spectral content, and/or spatial location within the audio field of the full pattern or at various parts of the pattern.

According to another aspect of the method, the user's movement is evaluated and the full pattern or parts of the pattern of the reference rhythm are modified to be in synchronization with the user's movement. For example, the rhythm of the user's movement is determined and the music and/or beat signals are modulated to synchronize with the user's movement.

According to yet a further aspect of the method, the rhythm may be variable or fixed. The music and/or beat signals can be are supplemented with additional beat signals or music to emphasize specific parts of the pattern. For example, the music and/or beat signals can be supplemented with additional beat signals or music to transform the variable pattern to a fixed pattern.

According to still a further aspect of the method, the modified rhythm is set back to reference rhythm after the user has returned to the reference point movement. The method continues to monitor the user's movement and modify the rhythm of the music each time the user moves away from the reference point movement. Examples of movement that the method may evaluate include, but are not limited to, walking, running, leg exercises, arm exercises, leg movements, arm movements, trunk movements, and weight lifting. It is also possible to use this method in speech therapy in order to assist a person in regaining or improving speech or voice.

According to another aspect, the method may be divided into sessions, wherein a session includes a number of movements by the user or a distance gained by the user, whereby the method includes continuing to monitor the users movements, continuing to alert the user that the user's movement is away from the reference point by modifying the reference rhythm, maintaining the modified rhythm until the movement is corrected and altering the rhythm back to the reference rhythm after correction of the user's movement, whereby these steps are repeated until the session is complete. In one preferred embodiment, a session comprises a distance of fifteen feet; wherein the session is directed at correcting an incorrect gait, and wherein the gait of the user is improved by about 1 to about 20 percent after one session.

According to a further aspect of the method, the reference point of movement is determined from a medical database including patients' medical records, patients' medical data, and/or patients' biofeedback data. Additionally, the reference point of movement may be determined from the user's physical state, textbooks, reference manuals, and/or the user's disability.

According to still another aspect of the method, the reference point of movement includes the ultimate movement goal to be achieved or one of many movements necessary in order to achieve the ultimate movement goal.

According to yet another aspect of the method spoken instructions may be provided to the user in addition to the reference rhythm. The spoken instructions include movement instructions to assist the user in performing the correct movement. For example, instructions to assist the user in walking correctly may include “heel-toe” in repetition. Other examples for other types of movement include, but are not limited to, “up-down” in repetition, “left-right” in repetition, and “in-out” in repetition.

According to a further aspect, the method and all the aspects described herein can be used in assisting a person regain or improve his speech wherein a method for evaluating speech of a user and providing biofeedback includes setting a reference point for speech by the user, whereby the reference point corresponds to a reference rhythm, providing the reference rhythm to the user to assist the user in maintaining the reference point; sensing the user's speech, comparing the user's speech to the reference point rhythm; and alerting the user that the user's speech is away from the reference point by modifying the reference rhythm to a modified rhythm.

It is another embodiment of the present invention to provide a system for evaluating movement of a user and providing biofeedback, wherein the system includes a component for providing rhythm to the user, a component for setting a reference rhythm, and one or more sensors for sensing movement of the user, wherein the component for providing rhythm alters the reference rhythm upon incorrect movement of the user to alert the user to the incorrect movement, and wherein altering the reference rhythm comprises modifying the reference rhythm to a modified rhythm. Moreover, in the system, the reference rhythm is modified by increasing or decreasing the amplitude of the rhythm.

According to another aspect of the system, the reference rhythm comprises music and/or beat signals. It is preferable that the music and beat signals are in synchronization. The music can include rock, pop, classical, jazz, hip hop, blues, alternative rock, rap music or a combination thereof. The beat signals can include sounds from a musical instrument or a metronome or a combination thereof. Examples of musical instrument include drums, symbols, a wind instrument, a string instrument, a piano, a harpsichord, an organ, or a combination thereof.

According to yet another aspect of the system, the amplitude is increased by increasing the amplitude of the music, the beat signals or both and the amplitude is decreased by decreasing the amplitude of the music, the beat signals or both.

According to a further aspect of the system, the rhythm can be further modified by increasing or decreasing the tempo, timbre, frequency, pitch, spectral content, and/or spatial location within the audio field.

According to yet another aspect of the system, the modified rhythm is set back to reference rhythm after the user has returned to the reference point movement. The systems continues to monitor the user's movement and modify the rhythm of the music each time the user moves away from the reference point movement. Examples of movement that the method may evaluate include, but are not limited to, walking, running, leg exercises, arm exercises, leg movements, arm movements, trunk movements, and weight lifting.

According to another aspect, the system can provide treatment in the form of sessions, wherein a session includes a number of movements by the user or a distance gained by the user, whereby the system includes continuing to monitor the users movements, continuing to alert the user that the user's movement is away from the reference point by modifying the reference rhythm, maintaining the modified rhythm until the movement is corrected and altering the rhythm back to the reference rhythm after correction of the user's movement, whereby these steps are repeated until the session is complete. In one preferred embodiment, a session comprises a distance of fifteen feet; wherein the session is directed at correcting an incorrect gait, and wherein the gait of the user is improved by about 1 to about 20 percent after one session.

According to a further aspect of the system, the reference point of movement is determined from a medical database including patients' medical records, patients' medical data, and/or patients' biofeedback data. Additionally, the reference point of movement may be determined from the user's physical state, textbooks, reference manuals, and/or the user's disability.

According to still another aspect of the system, the reference point of movement includes the ultimate movement goal to be achieved or one of many movements necessary in order to achieve the ultimate movement goal.

According to yet one more aspect, the system may include one or more of an interface component linked to the component for providing rhythm, a transceiver for receiving an transmitting signals, an analog digital converter, a storage device, a digital signal processor, a database, wherein the transceiver is linked to one or more of the sensors, interface component, an analog digital converter, wherein the analog digital converter is linked to one or more of the sensors and digital signal processor, wherein the digital signal processor is linked to one or more of the analog digital converter, storage device and interface component, wherein the storage device is linked to one or more of the interface component, and a digital signal processor, and wherein the database is linked to one or more of the interface component, the transceiver and the sensors.

### BRIEF DESCRIPTION OF THE DRAWINGS

Embodiments of the present invention will be more fully understood and appreciated by reading the following Detailed Description in conjunction with the accompanying drawings, in which:

FIG. 1 is a flowchart illustrating steps associated with an example embodiment.

FIG. 2 illustrates hardware components that are included in a preferred embodiment.

FIG. 3 is a flowchart illustrating steps associated with an example embodiment.

### DESCRIPTION OF THE EMBODIMENTS

Present invention: means at least some embodiments of the present invention; references to various feature(s) of the “present invention” throughout this document do not mean that all claimed embodiments or methods include the referenced feature(s).

To the extent that the definitions provided above are consistent with ordinary, plain, and accustomed meanings (as generally shown by documents such as dictionaries and/or technical lexicons), the above definitions shall be considered supplemental in nature. To the extent that the definitions provided above are inconsistent with ordinary, plain, and accustomed meanings (as generally shown by documents such as dictionaries and/or technical lexicons), the above definitions shall control. If the definitions provided above are broader than the ordinary, plain, and accustomed meanings in some aspect, then the above definitions shall be considered to broaden the claim accordingly.

To the extent that a patentee may act as its own lexicographer under applicable law, it is hereby further directed that all words appearing in the claims section, except for the above-defined words, shall take on their ordinary, plain, and accustomed meanings (as generally shown by documents such as dictionaries and/or technical lexicons), and shall not be considered to be specially defined in this specification. In the situation where a word or term used in the claims has more than one alternative ordinary, plain and accustomed meaning, the broadest definition that is consistent with technological feasibility and not directly inconsistent with the specification shall control.

Unless otherwise explicitly provided in the claim language, steps in method steps or process claims need only be performed in the same time order as the order the steps are recited in the claim only to the extent that impossibility or extreme feasibility problems dictate that the recited step order (or portion of the recited step order) be used. This broad interpretation with respect to step order is to be used regardless of whether the alternative time ordering(s) of the claimed steps is particularly mentioned or discussed in this document.

As will be appreciated, an embodiment of the present invention provides a process for providing biofeedback to a person performing a movement of his or her body. The movement can be of any body part. For example, the movement of a person's legs during the process of walking can be analyzed using the process herein. A person who has been injured or who has a physical disability or disease may require rehabilitation. The process herein may be used to monitor the person's walking and provide feedback to assist the person in correcting or adjusting incorrect movements occurring during walking.

The process typically begins by setting a target or reference movement that the person would like to or is required to achieve. For example, a stroke victim may have a limp on one side, which was created by the stroke. In order to overcome the limp, a target or reference movement will be normal walking without a limp. This reference movement is programmed into a hardware component such as flash memory or a software database contained in the device. This hardware and software database can be updated manually, via a button or automatically, such as in the case of a patient walking into a doctor's office, which wirelessly updates the database. Depending on the person's injury or disability, the target movement may not initially be the end result (e.g., full movement with no limp) that will eventually be achieved, but will be adjusted or calibrated periodically as progress is made, to eventually attain the end result (e.g., full movement with no limp). The target movement will be continuously updated as progress is made. In this way progress may be made gradually in order to not overstress the muscles, bones or other body part being rehabilitated.

After the target or reference movement has been set, the person may begin rehabilitation. Sensors are placed on the parts of the body that will be monitored for movement. For example, if a person needs correction or rehabilitation in the muscles of his legs, sensors will be attached to his legs and/or feet in various locations to measure the movement of these body parts. In addition to sensors, the person will be provided with music to listen to, which music will have a certain tempo, rhythm, beat, pitch, timbre, spectral content (i.e., equalization), and/or spatial location (i.e., balance) within the audio field. Examples of types of music that may be used, include but are not limited to, rock, classical, jazz, hip hop, blues, alternative rock, rap or a combination thereof.

The music can be provided by either hardware, software, or any combination thereof contained in an MP3 player such as an iPod® device, compact disc player, or other portable device. It is preferable that the music is in a mobile format that can be carried or easily attached to the person by a carrier or other means. At the beginning of the session, the music is provided at a specific tempo, rhythm, beat, pitch, timbre, equalization, and/or balance, which is considered the reference or target music corresponding to the target movement. It is preferable that this auditory cueing method improves common neurologically impaired behaviors, e.g., gait, by setting the tempo at a pace that is close to or matches that of the patients' normal movement pace.

The sensor or sensors attached to the person are linked to the music in such a way that the reference music corresponds to the reference movement. That is, if the person's movement simulates the reference movement, the reference music will play. If the person begins to move away from the reference movement, or incorrectly, the music will be altered from its reference sound in one or more ways, such as by increasing or decreasing the tempo, rhythm, beat, pitch, timbre, equalization, and/or balance of the music in order to alert the person that his movement is incorrect.

In a preferred embodiment, the music is altered to alert the person by increasing or decreasing the amplitude of the rhythm. This can be done by increasing or decreasing the volume of the rhythm. By increasing or decreasing the volume, the person will recognize the change in the music and will be alerted to change his movement back toward the reference movement. Once the reference movement is achieved, the rhythm can be readjusted to its original or reference state. The frequency of the rhythm can also increase or decrease in order to produce a more audible rhythm.

This biofeedback will adjust the music output in response to suboptimal movement, such as an insufficient heel-strike during walking. This biofeedback data will be referenced through a database of normative movement values. These values can refer to any movement, such as normative gait parameters. As soon as the user moves incorrectly, such as striking their heel too softly during walking, the device will record this walking as well as deliver output to the user in real-time.

In a further embodiment, another way to alter the rhythm is by adding an additional beat signal that is in synchronization with the original rhythm. In this way the original rhythm is amplified by the addition of signal beats. The rhythm is the same pattern, but is amplified at certain movements of the pattern, thereby maintaining the same rhythm, but with added sound. The beat signal may be provided in the form of drums, a metronome, symbols, a wind instrument, a string instrument, a piano, a harpsichord, an organ, or a combination thereof. By increasing the amplitude of the rhythm with different sound, but the same beat, the person hears the change in the music and alters movement towards the target movement. Additional signals or channels can be added in order to produce distinct audible references that convey additional rhythmic information. For example, changes in amplitude, volume, pitch, or the like, can be added at ¼th note, ⅛th note, 1/16th, etc. . . . to make the song's reference signal more tightly matched to the rhythms or the patient's ongoing activity. So if they have a gait with a downbeat: ONE-two-THREE-four, the music, which has an upbeat: one-TWO-three-FOUR—can be altered to match their specific rhythm.

In yet another embodiment, the music and the beat signal may begin together as reference music. If the person's movement moves away from the reference movement, the beat signal may be decreased or increased in amplitude or frequency to alert the person of incorrect movement or the music may be decreased or increased in amplitude to alert the person of incorrect movement.

The method is able to adjust music in real-time in response to biofeedback received from the sensors. The increase of the rhythm not only alerts the person that his movement must be altered, but the pronouncement of the beat and rhythm may assist the person in “keeping beat” with the rhythm.

The simultaneous output of music and tempo can occur in a single channel or in separate channels. It is preferable that the output of the music and beat duplication must occur in synchrony. Accordingly, the beats of the beat signal output must occur at the same time as the beats playing in the music, (within a few milliseconds). The method can include the option to play numerous songs in synchrony according to their tempo or any music property and alter their output by using a sensor or sensors that record physiological movements. In the case of a patient walking incorrectly, a patient listening to a song via earbuds, speakers, or the like, an additional song will begin to play in synchrony with this song. This additional song, channel, or sets of audio channels will alert the patient that they are walking, sitting, talking, or the like, incorrectly. This sort of integration can be seen in many common disc jockey (DJ) mixes, in which popular songs are mixed with another song or songs to create a DJ remixed version of the songs. Unlike existing manual approaches, which often take DJ's many hours of mixing and matching of songs, this unique approach automates the process using any sensor or group of sensors that generate audio output via physiological movements. This biofeedback approach is unique because it enables patients to improve their health while they are listening to music that plays in synchrony with any number of reference signals.

The output of the audio can also be set up to controlled by the user's cadence. Should the user begin to move too slowly, (such as walk slowly or even stop walking), the output of the tempo of both channels will be controlled by the user's cadence (beats per minute) or any other physiological movement. For example, if the user abruptly stops walking, the output of the device will stop. Moreover, the output of the beat signal channel can be either auditory or tactile (such as an electrical stimulation or vibratory).

The movement values detected by the sensor of this device can refer to any movement, such as normative gait parameters, which include by are not limited to cadence, velocity, step length, cadence, and stride symmetry. Any number of sensors might be suitable to the task of measuring a given movement.

It should be mentioned that the audio element can be provided by an external source or an internal source, such as software that integrates with an MP3 player. The extraction of and duplication of rhythm will increase or decrease the volume of the beat signal and/or music in response to any sensor or group of sensors that measure physiological movements. Examples of additional audio cues that can be injected into the music or signal beat currently playing might occur in quarter and/or half-notes in any music's rhythm. The audio cues can be high or low in pitch/volume, bass, or the like. The goal is to provide cues that will help patients move to the contour of these cue injections. The reason is because many tempos in music are perceived by listeners by the lows and highs that occur before or after each beat. For example, some music is more upbeat, including pop music, and some music is heavy in its beat, such as music from marching bands. The goal is to make music that might be heavy in its rhythm to be more upbeat, which is useful to promoting physiological behaviors that require anti-gravity movements, such as toe-off in human walking. Hardware/software analysis and duplication of rhythm (i.e. the rhythm or tempo extraction and duplication) can occur within an MP3 player's software, between the MP3 player and the earphones, or at the point of wireless receiver plugging into the MP3 player. Also, this rhythm or tempo extraction and duplication can occur in real-time or offline.

In altering the intrinsic rhythm of the music, a song's tempo can be altered to match the tempo of a person's gait by adjusting the intrinsic rhythm. That is, a song will keep playing at the SAME SPEED but will make the tempo sound slower/faster by overlaying a metronome in a unique method. This method involves making the louder beats occur when a person, for example, strikes his heel. Accordingly, the song stays the same speed but the tempo gets changed by (i) calibrating the reference signal to the person's movement and (ii) making the reference signal play with the song's unadjusted tempo. The only difference is when the new tempo's high points are audible.

In addition to rhythm, tempo may be modified to assist the user in keeping pace with the target rhythm. As is known, fast paced songs tend to prompt listeners to move quickly. Conversely, slow songs tend to prompt listeners to move slowly. This is particularly important when setting the reference music. The tempo should be taken into consideration to assist the user in maintaining the target movement. Accordingly, the tempo of the output can be increased or decreased based on the speed of the user's movement, such as walking. In the method herein, the person's “normal” movement (e.g., gait, walking up steps, jogging, etc.) can be identified and the rhythm can be slowed down or speeded up in tempo to improve the person's behavior. This “smart” response improves the person's sensorimotor coordination.

Additionally, the tempo can be altered based on the speed of the movement by the user. For example, if the movement is slow, the music, which might normally have a tempo of 60 beats per minute, might slow down to 30 beats per minute. The beats per minute will be controlled by a physiological movement, which is detected by the sensor(s). The tempo will be altered to match the input collected from a given sensor representing a physiological movement. Thus, a fast song, such as the Beatles' Twist and Shout, will slow down to a much slower rate in the case of the person moving slowly. In this case, the slowing of the output will have occurred in real-time. This can be a separate biofeedback element, which can serve to assist the user in keeping with his target movement, by providing optimal tempo which matches the speed or tempo of the movement of the user. By slowing down or speeding up the reference music to the speed of the movement by the person, movement is made easier, providing motivation and incentive to maintain correct movement.

In one embodiment, the tempo of the output will increase or decrease manually, based on a determination made by the user or by the therapist. This determination will identify a safe movement speed for the person undergoing physical therapy at which to move. The tempo will be altered to match the input collected from a given sensor representing a physiological movement. Thus, a fast song, such as the Beatles' Twist and Shout, will slow down to a much slower rate in the case of a user or therapist adjusting the speed output. In this case, the slowing of the output will have been predetermined.

In an alternate embodiment, the tempo of the output will increase or decrease by a normative value-based function included in the software. An example of this normative value-based function will receive input from an online network that collects the rehabilitation progress of various persons using this technology, and determines an optimal tempo speed based on an assessment of the individual's progress. This function will identify normative movement parameters, including walking speed, and set the music according to this movement.

In the case of the output playing too slow or too fast with respect to the movement parameter being measured, the song can play extra slow, the user can select a different song, or the device can automatically select a different song matching the tempo of the physiological parameter being measured.

Sensors suitable to the task of measuring movement parameters include, but are not limited to, accelerometers placed on an arm, leg, torso, head, or the like. In order to more finely measure the movement of the body additional sensors can be utilized, such as electromyogram (EMG) sensors, which measure muscle activity, or even internal biosensors, which interface with internal biochemical processes of a person. Moreover, a gait initiation mechanism may be included whereby the sensor(s) detect a freezing in gait and prompt rhythmic stimulation via any number of auditory, tactile, or vibratory methods.

In one embodiment, a stimulation mechanism is included with the sensor that, upon detecting “freezing,” delivers an auditory, tactile, or combination of cueing methods that cues the person to move forward from a “freeze.” In an auditory embodiment, the device stops playing music and the beat signal for a brief moment, then outputs a sound in one ear and then a sound in another ear. Additionally, tactile or vibratory stimulation components placed on orthotics deliver tactile cueing to prompt the user to walk forward. The moment one step is taken, another rhythmic beat or tactile stimulation can take place on the opposite side of the body to prompt the right side to walk forward.

Biofeedback herein can refer to a signal that can be visual, auditory, or tactile (physical stimulus), ant hat is used to modulate ongoing behavior. The method herein will rhythmically train persons with significant sensorimotor deficiencies to regain motor control.

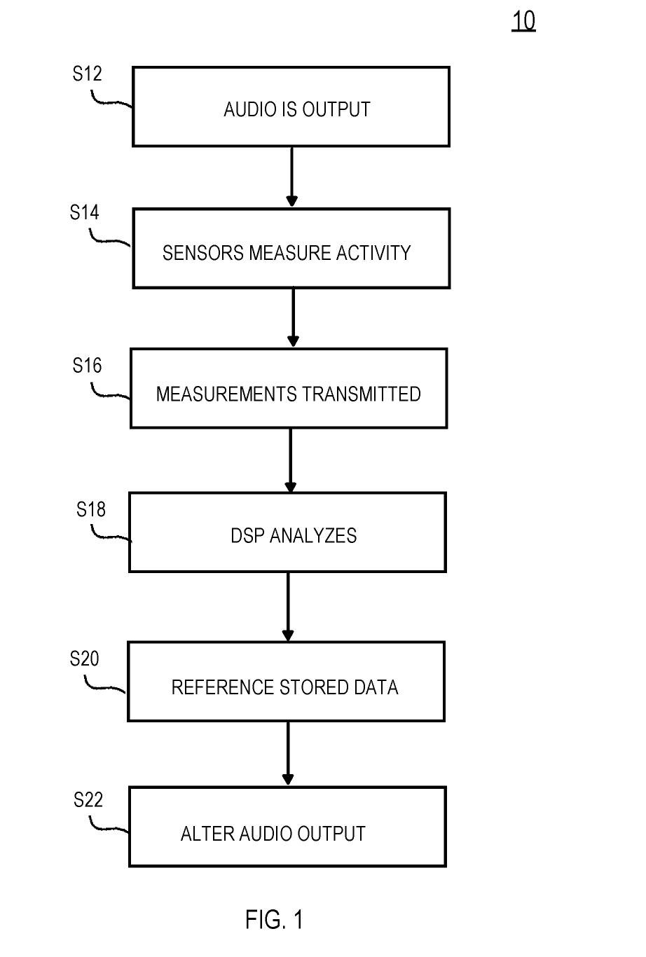

Reference is made to FIG. 1, which shows a method 10 in the form of a flowchart illustrating example steps associated with a preferred embodiment. At step S12, music plays from a music player for the user's enjoyment. As the user moves, sensors measure physical activity at step S14. Information from the sensors, in the form of digital, analog signals or a combination of the two, is transmitted via wireless (e.g., via BLUE TOOTH) technology to a receiving unit at step S16. The analog signals from sensors may be converted via an analog digital conversion process into digital signals and the digital signals are transmitted wirelessly thereafter. Once the signals are received, they are analyzed at step S18. The received signals are compared with the reference data at S20 to determine whether the signals received in step S16 indicate whether the user is complying with a predetermined reference movement. At step S22 the audio output is altered if the signals received in step S16 do not match the reference data.

In another embodiment herein, a system is provided to assist persons needing correction or rehabilitation to improve physical limitations, such as those associated with restricted range of motion and other impairments associated with physical conditions, including neurological conditions or traumatic injuries. A person's physical rehabilitation is rapidly accelerated and improved by use of biofeedback in connection with music. The system includes reference music, sounds and instructions which corresponds to a target movement of the user of the system. The target movement is that movement that the user is trying to attain. For example, the user has a limp that he is trying to overcome. The target movement is walking without the limp. The system includes a component with a catalogue of reference movements, i.e., correct forms of movement performed by an average person having no physical disabilities or limitations. This catalogue of reference movements may interact with a single pool of data included in this device or with external pools of data, such as those included in an online database in real-time. Such an online database could take place, for example, in a doctor's office, where an internet connection is secure and reliable. The reference movements may be modified to cater to the user, by a person having expertise in this area, such as a physical or occupational therapist. For example, if a user's baseline or normal walk (prior to injury or disease) has an under or over pronation, a reference movement sample in the catalogue could be modified to include an under or over pronation. In the case of a limp, the device references to a pool of reference movements and activates a sound or a series of sounds such as a modified tempo that corresponds to the limp. This tempo could, for example, increase the frequency or amplitude in the earphone that is on the same side as the impaired leg in order to deliver an auditory reference that is tailored to the person's movements. As mentioned previously, this reference continuously updates and alters its output, such as decreasing the audible limp, in order to correct the impaired movement.

Additionally, a new reference movement could be added to this component to act as the target movement for the user. Once the target movement is determined, the music must be selected and calibrated to the target movement. Any type of music can be used for the target movement, as long as the system is calibrate the reference movement to the reference or target music. The reference movement and music can be determined manually by a patient or by a clinician. For example, the patient or clinician can touch a button or speak into a microphone to select a target movement and reference music. The music can also be organized into categories according to their tempo—slow songs would be organized into a level appropriate for someone with very limited range of motion, such as a slow gait. The music can also be selected by the patient (such as it is in any M3 player) and it is then adjusted with respect to reference signal (i.e., tempo).

The system further includes a music player to provide music to the user of the system. The music can be in the form of a stereo, compact disc player, sound system, MP3 player, Ipod® device, or other portable device. Music is preferably provided to the user via headphones, ear buds or the like, although it is also possible to provide music by speakers or a sound system. The music component is linked to sensors either by wire or by wireless devices. Sensors may be placed anywhere on the person or user, preferably on a location that measures electrical activity of the user's muscles.

The music is altered in terms of volume and/or rhythm as a function of the measured physical activity of the user's muscles. The purpose is to make any music useful to delivering rhythmic auditory stimulation (RAS). For example, EMG sensors that measure electrical activity of the user's muscles can be used. If the user needs to modify some behavior, such as a rhythmic behavior, with respect to the reference movement, such as to improve his gait, then the audio output (e.g., the music) is altered in some way until the user makes the modification, such as in its rhythm, by enhancing the volume of the rhythm or frequency of the tempo.

In one embodiment, one or more suitable forms of sensor devices, such as biosensors and flexion sensors, measure muscular activity while a user walks. Flexion sensors may be preferable for having a relatively precise measurement of a user's mobility. For example, flexion sensors have a fairly consistent readout of movement, unlike biosensors, which may have some degree of variance. In addition to biosensors and flex sensors, any suitable sensor may be used in connection with the teachings herein that are operable to collect data from a user, particularly related to a user's mobility.

In another embodiment, flexion sensors are embedded in fabric such as spandex and placed on a user's body on for example, the knee in order to measure a degree of which the user is bending or flexing his knee. The sensor devices may be strategically inserted in various clothing worn by the user. Further, sensors may be placed in various clothes, such as in shoe soles to detect and record sub-optimal walking. Analog signals representing the detection are sent to a receiving device in real-time. The sensor measurements are used to provide signals that are transmitted wirelessly to a music playing (or other) device. The music-playing device preferably appropriately alters the audio output in response to the signals. When the user is not complying with a predetermined therapeutic practice, such as walking with a proper gait, the information detected by the sensors is used to alter the audio output of the player and alert the user accordingly. In one embodiment, as the sensors detect incorrect activity by the wearer, such as an improper walking gait, the signals cause the player to increase or decrease the volume.

In an alternative embodiment, the signals cause the audio to distort or otherwise be modified. For example, various audio characteristics or one or more audio effects of the audio output are altered, including pitch change, compression, distortion, flanger, phaser, delay or the like. Upon recognizing a change in volume or distortion in audio output, for example, the user uses the audio output as an indicator that the user's behavior or activity requires adjustment. When the user modifies his behavior, for example, by improving his walking gait, the audio resumes its undistorted or previous volume setting. Thus, the user can determine, simply by a change in the audio, that the user needs to take some corrective physical action. Physical rehabilitation or physical therapy is effectively reinforced as a function of the altering audio content, and the user develops a learning process whereby physical therapy and/or physical rehabilitation becomes intuitive.

In one embodiment, a musical song is used for the musical output. As noted above, the musical song either distorts or incurs some other change, such as a change in volume, when sensor measurements indicate that corrective measures need to be taken by the user. In one example, the audio gets substantially softer to the point that the user can no longer hear the audio when the user is not complying with the predetermined rehabilitation or physical therapy. Upon taking corrective measures, the audio volume resumes its previous level and the user can enjoy the music. In an alternative embodiment, the audio content gets significantly louder, thereby alerting the user that corrective measures are required. In another embodiment, a rhythm track, such as a drum beat or a combination of drumbeats is used for the audio output. The tempo of the rhythm adjusts as a function of the sensor readings. Accordingly, beats increase in amplitude or change in tempo (i.e., slow down or speed up) in connection with the sensor readings and corresponding signals.

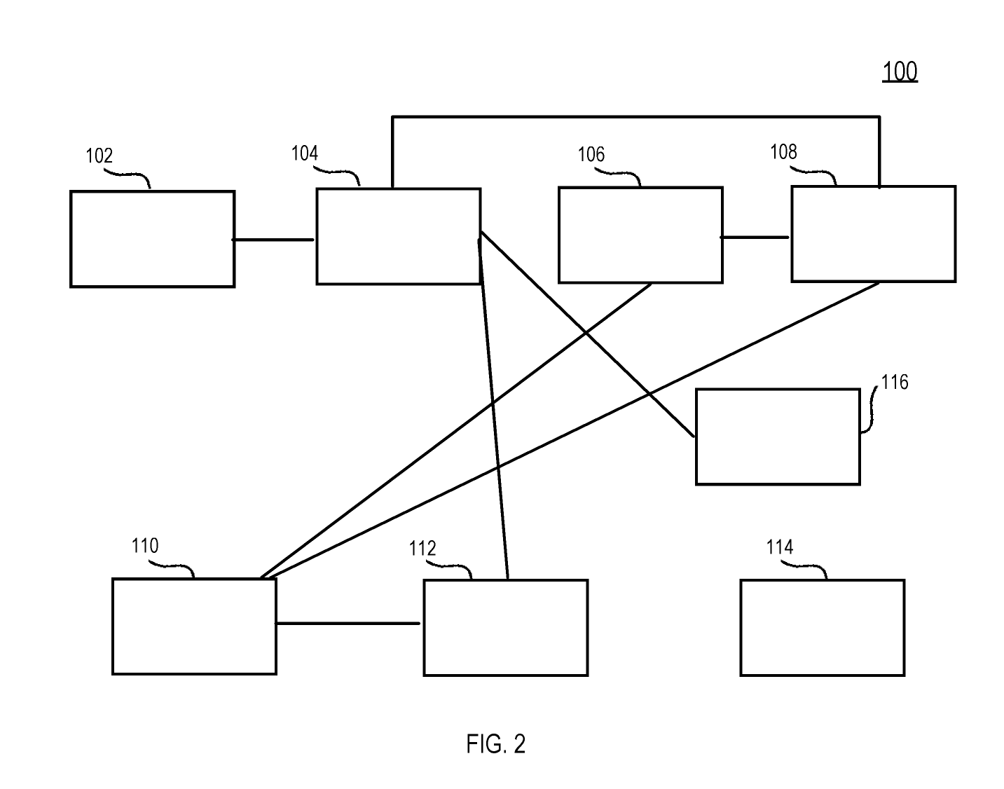

Reference is now made to FIG. 2, showing biofeedback system 100 preferably used to provide the biofeedback features herein described. System 100 includes a player 102 that outputs audio to a user. In one embodiment, player 102 is an MP3 player or other audio device that is operable to output digital audio files that are provided in various formats, such as WMA, WAV or other suitable format. Player 102 is preferably provided with an interface 104 that enables player 102 to alter the audio output, in accordance with the teachings herein. As shown in FIG. 1, sensors 106 are provided to track and/or measure electrical activity associated with muscle movement. Sensors 106 are preferably placed on, or adjacent to, one or more target muscles to obtain a high degree of precision with respect to tracking mobility and movement of the muscle. In one embodiment, sensors 106 preferably measure muscle activity and transmit signals that are received via one or more transceivers 108. Of course, one skilled in the art will recognize that various configurations are envisioned herein, such as using a transmitter to send sensor measurement signals to a receiver. Analog to digital converter 110 preferably converts the analog signal of the sensors 106 to digital signals. Analog to digital conversion (ADC) can happen prior to transmission or after it; or if sensors have built in ADC, then there is no need for ADC; or if sensors are digital there is no need for ADC. In one embodiment, analog to digital converter 110 converts signals prior to signals being transmitted from sensor 106. Alternatively, signals are converted from an analog to a digital format after being transmitted from sensor 106. In either case, the digital signals are interpreted to cause player 102 to manipulate audio output, as appropriate.

Digital signal processor 112 preferably analyzes signals received from analog to digital converter 110 and facilitates the manipulation of the audio output from player 102. For example, in case a user suffers from an anterior cruciate ligament (“ACL”) disorder, digital signal processor 112 recognizes patterns of improper walking and modifies the audio output from player 102 accordingly. Storage device 114 is preferably included and used to store data, including predetermined signals that are referenced by digital signal processor 112 to determine whether audio output from player 102 should be altered. Further, music or other audio content for player 102 may also be stored on storage device 114 and signals from audio to digital converter 110 may also be stored thereon. By storing signals representing a user's muscular activity, a user's progress can be tracked and monitored over time. In a preferred embodiment, storage device 114 interfaces (via interface 104) with a computer or other device 116 operable to receive data and perform various tasks, such as graphing statistical analysis or the like, or to alter programming of digital signal processor 112. In a preferred embodiment, storage device 114 is formatted as a solid state drive, such as a flash drive. Moreover, interface circuitry 104 preferably enables various components to be connected and operable with player 102. For example, storage device 114, digital signal processor 112 and/or analog to digital converter 110 interface with player 102 via connection interface 104. Connection interface 104 may be formatted, for example, as a circuit board, which is provided in a housing to be connected directly to player 102. Further, transceiver component 108 preferably operates with sensors 106 and/or interface 104 to wirelessly receive signals and operable with player 102.

In operating system 100, music is played from player 102 to a user. As the user moves, sensors 104 measure physical or electrical activity of the user or user's muscles. Information from the sensors, in the form of digital or analog signals, is transmitted by wireless (e.g., BLUE TOOTH® technology) technology to receiving unit. Alternatively, the analog signals from sensors 104 are converted via an analog digital conversion process by analog digital converter 110 into digital signals and the digital signals are transmitted to digital signal processor 112 for analysis. Preferably, digital signal processor 112 references data stored on storage device 114 to determine whether the signals received indicate the user is complying with a predetermined reference. Digital signal processor 112 uses the data and signals and alters the music accordingly to affect audio output from player 102.

Thus, as shown in connection with FIG. 2, a plurality of components are preferably arranged in order to collect data from a user that represents the user's physical movements and mobility and used to translate those data into signals that can be interpreted and used to modify audio output in connection with biofeedback.

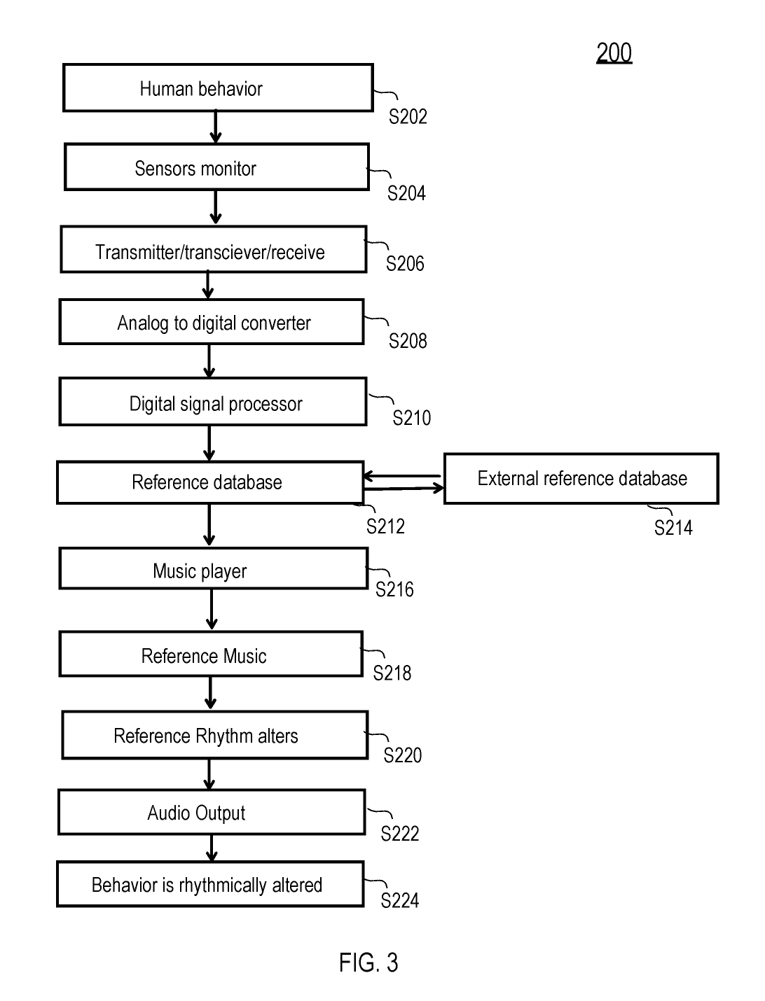

Reference is made to FIG. 3, which shows a method embodiment 200. The method begins with the user's behavior at step S202, which needs improvement or correction. At step S204, sensors monitor the user's movement. A transmitter receives the signal from the sensors at step S206 and sends it to an analog to digital converter at step S208. At step S210, a digital signal processor processes the signal and sends it to a reference database at step S212. An external reference base may be linked to the reference database and can provide reference movements to the reference database at step S214. The reference database is linked to a music player at step S216. At step S218 reference music plays from the music player. At step S220, the reference music is altered to correct the user's movement. At step S222, audio output is sent to the user and at step S224, the user's behavior is rhythmically altered.

In accordance with teachings herein, a user suffering from movement or other range of motion disabilities or disorders benefit by an intuitive combination of elements and tools that will assist in the user regaining or improving coordination and movement. For example, users suffering from forms of dementia, Parkinson's Disease or other conditions that disturb a user's central or peripheral nervous system and resulting in an inability to properly coordinate movement can benefit from the teachings herein. By using music for an output, users can use rhythms and other elements of the music to improve basic motor functions. Further, the embodiments of the invention are designed to be integrated in a user's clothing and do not interfere with the user's ordinary activities. In this way, users can regularly and consistently use the biofeedback therapy and improve the ability to recognize and correct improper behavior, such as walking with a poor gait. The use of “smart” continuous learning capability of the reference signal and music that 1) calibrates to the normal movement of the user and 2.) automatically alters its output to elicit an improved response provides improvement in the user's behavior. In the case of rhythm's speed and amplitude modulation, the speed and amplitude of the rhythm are increased to prompt users to increase their gait cadence and gait coordination. This is based on a method whereby clinicians deliver RAS at a pace that matches the patients' gait and increases the tempo in order to prompt them to improve cadence.

Further, cost savings will be realized in the healthcare industry as users' conditions improve over time by the regular use of the teachings herein. Therefore, the healthcare industry, which is currently experiencing staggering increases in costs, will benefit from the present invention. This is in part due to the ability for users to continue muscle training and improvement even while away from a physical therapy clinic or center. As changes in audio output occur, the user becomes aware of a need to take corrective action in connection with a particular motion. Accordingly, the present invention reduces both the time and money typically required in the prior art for users to fully recover from an injury and/or to improve a chronic condition.

In another embodiment, a system is provided for compiling patient profiles in order to gauge patients' overall health. This compilation of a patient profiles can be achieved in any number of ways. An example of fashioning a patient profile includes but is not limited to extracting information from hospitals' existing information systems and assembling a set of data that creates a patient's health profile. This extraction would enable this system to interface with web applications as well as desktop applications in a behind-the-seams, easy-to-use fashion. The compilation of a patient profile can also be entered manually. It can also be compiled by utilizing medical devices that aggregate data via diagnostic, therapeutic, and monitoring processes. For example, diagnostic device described herein include the ability to automate the assessment process whereby clinicians gather relevant health information in order to gauge their overall health. During the process of automating a process, such as asking a patient questions relevant to their health, the device can transmit this data to the system automatically, manually, or in any way that is deemed convenient, useful, and respectful of privacy by clinicians. This data can also simply remain within a device that is part of the system described herein. In other words, the system can be integrated via computers, devices, and the like. Another example of a medical device gathering relevant information is in the case of an at-home monitoring device that monitors a patient's health, which can be stored in a device and integrated into the system described herein. This integration can be done so manually, wirelessly, over the internet, via flash, or in any means that is deemed useful and convenient by clinicians.

The integration of devices, portals, pools of information, and the like, can be accomplished through an internet cloud architecture. An architecture is provided whereby health information is stored and hosted on a number of servers on the internet and is cached on client computers. Other options include a single server or limited number of servers can host relevant information and devices and portals can access this information from their respective locations; peer-to-peer sharing networks; and applications that are not web-based but interact in various ways with information that is downloaded to the system via the web. This system's architecture includes any means of establishing an information interface. The architecture itself might be solely web-based, solely desktop application-based, or it may include a combination of the web and desktop application structures.

The system extracts or refers to pools of data pertaining to health information in order to compare it to a patient's health profile. This extraction, reference, cross-analysis, and the like, of data includes, but is not limited to, demographic information, information relating to clinical research, financial information, or any information relevant to the analysis of a patient's health. This extraction or reference can occur from a hospital's existing EMR system, such as from the hospital's billing and documentation software. This software is useful to the task of data extraction or reference because it includes relevant health information and codes relating to the treatment rendered by clinicians.

Data can also be extracted or referenced from a pool of demographic information, which includes databases from an external source, such as those from insurance companies. Any pool of data, either internal or external to the system described herein, can be extracted, referenced, cross-analyzed, or the like, and can be used for any purpose relevant to the task of treating patients. The decision to extract or reference a particular data pool can be either automated or manual. For example, a clinician may select the types of data pools they are interested in, including but not limited to databases with academic journals, demographic information, and the like. This pool of data may or may not be from existing EMR systems in given hospitals.

The system described herein is capable of providing assistance to clinicians when they are treating patients. Clinicians will benefit from this embodiment because they often are expected to follow protocols—standardized processes—when treating patients. This embodiment enables clinicians to simply follow steps that are provided on this system via visual and/or auditory cues. For example, a voice can dictate the steps the clinician needs to follow in a clinical activity, such as during assessment or diagnosis. The voice would provide instructions or cues that help the clinician follow protocol with greater ease and efficiency. Alternatively, this cueing is simply delivered via text or iconography, such as for example, by a device displaying the steps in a clinical protocol. Cueing could also be delivered numerically. This cueing—visual or auditory—can be delivered in any number of scenarios, including but not limited to diagnostic, therapeutic, and assessment activities.

The system is capable of extracting information from a patient profile that is compiled and finding the most relevant and respected journal articles in the evidence-based search portal for the patient. This external pool of data, which includes academic journal databases, is an important part of the system described herein. The system can include a portal whereby clinicians can search keywords to find the most relevant and respected journal articles. In an effort to help find the best articles, the system may automatically suggest keywords based on the patient's health profile or clinicians may enter in keywords. The system may also find articles based on factors pertaining to the patient's health profile. The methods whereby the system efficiently finds the most relevant and best articles include but are not limited to diagnostic methods that clinicians use, the overall quality of the patient's rehabilitation, the patient's health history, and the like. Any information and methods included in the patient's health profile may be used to find relevant and high quality journal articles.

In this embodiment, the system rates the quality of the journal articles to help clinicians find the best literature. For example, the system may rate a journal as high quality because it is double-blinded and randomized. In another example, the system includes a method whereby articles are rated as high quality based on the number of times they have been retrieved by clinicians. Any information and methods included in the patient's health profile may be used to find relevant and high quality journal articles.

In a preferred embodiment, a system is provided incorporating the database system having patient, hospital and medical information (“medical database”) into the biofeedback method and system described herein to provide reference data that may be useful for using with a patient. For example, if a patient has a certain ailment that results in improper form of the patient's gait, the therapist or doctor of the patient can utilize the wealth of information provided in the medical database to determine the best course of rehabilitation to follow by searching the database for similar symptoms, similar background (e.g., age, gender, weight, height, etc.) to provide the best protocol (frequency of treatments, number of treatments, number and range of movements needed to reach target movement, length of time for use of a reference movement before the next reference movement may be used, etc.). By availing therapists and doctors to the medical database, time and work are decreased reducing the costs of medical treatments.

In another embodiment, this system includes therapeutic devices that have diagnostic, therapeutic, and medical monitoring capabilities. For example, a portable music or phone device such as an iPod® device or iPhone™ device may include an infrared camera, either attached directly via hardware or software, or in an external attachment of hardware or software. This infrared camera will be useful to the task of visualizing pain. The portable device may have any medical technology either attached directly via hardware or software, or in an external attachment of hardware or software. Any medical device may integrate in the system described herein.

The system further includes the ability to deliver voice-feedback to patients in real-time. For example, when exercising or walking at home, the patient will hear feedback regarding their movements. Unlike many existing devices, this voice responsiveness occurs at the moment a particular movement occurs. An example includes if a patient walks incorrectly or sits with in incorrect posture, the device will deliver a voice that alerts the patient about their ongoing behavior. This audible feedback may be delivered via earbuds, speakers, or the like. In another example the system includes the ability to deliver reports to patients regarding their health after a set of movements have occurred. For example, after performing a therapeutic procedure, such as exercising or delivering an insulin shot, the device will deliver a report, that may say something regarding the overall procedure. The feedback includes anything relevant to the patient's health. In another embodiment, the system delivers audible feedback that includes non-voice-feedback. For example, after an exercise, a round of applause may delivered in the case of a therapeutic procedure being performed correctly. In the case of a patient performing a therapeutic procedure incorrectly, an alternative audible feedback may be delivered, such as booing. Any audible feedback may be delivered regarding ongoing behaviors or therapeutic procedures and methods.

In another embodiment the system includes the ability to take notes from the clinician. Clinicians often perform note-taking abilities with paper or on a laptop. This system includes the ability of the clinician to simply speak their notes, which will then be written electronically into the system described herein. The clinician may also write these notes down in a device, such as an iPod® device or iPhone™ device, which will incorporate notes into the system described herein. The clinician may speak their note-taking and at the same time, transcription may occur to save clinicians time later in the day, which is when they usually call a medical transcription professional to convert their spoken reports to text format.

The system can include a security component to prevent exposure of the information in any number of means. One example of achieving a securitized set of data includes the system stripping the person's name and sending this data to a server or set of servers to compare it to pools of information. Another example involves the system sending data to a double-encrypted server or set of servers. The system can include the ability to securitize information via any means that is deemed useful, convenient, and respectful of patient privacy by patients, caregivers, and clinicians.

### Example 1

Testing was performed using 6 elderly people who reported problems associated with gait. The device was used from a laptop demonstration using LabVIEW software available from National Instruments Corporation, Austin, Tex. and foot switches from B & L Engineering, Santa Ana, Calif. The footswitches include four units for each foot and were used to determine the foot/floor contact for gait analysis. Contacts for the foot switches were provided at the heel, fifth metatarsal, first metatarsal and great toe to indicate when these areas of the foot were bearing weight. The participants were measured walking on a 15 foot walkway without using the method and system herein (pre-measurement). The participants then walked down a 15 foot walkway using the method and system as discussed herein listening to the device as they walked and providing correction by altering rhythm, if movement went off-target. The device was then turned off and they walked once again 15 feet (post-measurement). The change in gait is shown in Table 1 below.

TABLE 1

| | Pre- | Post- | Percentage of |
|---|---|---|---|
| Participants | measurement | measurement | Change |
| 1 | 92.5 | 101.3 | 8.8 |
| 2 | 97.7 | 105.4 | 7.7 |
| 3 | 86.3 | 104.2 | 19.9 |
| 4 | 91.4 | 105.4 | 1 |
| 5 | 102.7 | 99.6 | −2.8 |
| 6 | 93.3 | 94.2 | 0.9 |
| Average | 93.9 | 101.6 | 8.1 |

Participants demonstrated an improvement in cadence as high as 19.9 percent and at an average of 8.1 percent after just one use for a short distance of 15 feet.

The innovation described herein uses biofeedback, music, and tempo to rhythmically train persons with walking impairments to regain coordination. Unlike previous biofeedback inventions, the technology described utilizes a unique method whereby the device's output includes music as well as a duplication (either performed in a software application, an external hardware application, or a manual process) of the song's tempo. This biofeedback will adjust the music and the tempo duplication output in response to suboptimal movement, such as an insufficient heel-strike during walking. This biofeedback data will be referenced through a database of normative movement values. These values can refer to any movement, such as normative gait parameters. As soon as the user moves incorrectly, such as striking their heel too softly during walking, the device will record this walking as well as deliver output to the user in real-time.

Although the present invention is described and shown in relation to particular embodiments thereof, many other variations and modifications and other uses will become apparent to those skilled in the art. For example, the invention is applicable to animals that are able to perceive audio tones. Further, young children, and even infants may benefit from the teachings herein. It is preferred, therefore, that the present invention be limited not by the specific disclosure herein.

What is claimed is:

1. A method for evaluating movement of a user and providing biofeedback comprising:
setting a reference point for movement by the user, whereby the reference point corresponds to a reference rhythm;
providing the reference rhythm to the user to assist the user in maintaining the reference point;
sensing the user's movement;
comparing the user's movement to the reference point movement; and
alerting the user that the user's movement is away from the reference point by modifying the reference rhythm to a modified rhythm.

2. The method of claim 1 wherein the reference rhythm is modified by increasing or decreasing the amplitude of the rhythm.

3. The method of claim 2 wherein the reference rhythm comprises a pattern of movements.

4. The method of claim 3 wherein the pattern of movements comprises music and/or beat signals.

5. The method of claim 4 wherein the music and beat signals are in synchronization.

6. The method of claim 5 wherein the amplitude is increased or decreased by increasing or decreasing the amplitude of the music, the beat signals or both.

7. The method of claim 6 wherein the amplitude is increased or decreased at specific parts of the pattern.

8. The method of claim 4 wherein the music comprises rock, pop, classical, jazz, hip hop, blues, alternative rock, rap music or a combination thereof.

9. The method of claim 4 wherein the beat signals comprise sounds from a musical instrument or a metronome or a combination thereof.

10. The method of claim 9 wherein the musical instrument comprises drums, symbols, a wind instrument, a string instrument, a piano, a harpsichord, an organ, or a combination thereof.

11. The method of claim 3 wherein the rhythm is further modified by increasing or decreasing the tempo, timbre, frequency, pitch, spectral content, and/or spatial location within the audio field of the full pattern or at various parts of the pattern.

12. The method of claim 11 wherein the user's movement is evaluated and the full pattern or parts of the pattern of the reference rhythm are modified to be in synchronization with the user's movement.

13. The method of claim 1 further comprising returning the modified rhythm to the reference rhythm after the user has returned to the reference point movement.

14. The method of claim 11 further comprising continuing to monitor the user's movement and modifying the rhythm of the music each time the user moves away from the reference point movement.

15. The method of claim 1 wherein the movement comprises walking, running, leg exercises, arm exercises, leg movements, arm movements, trunk movements, or weight lifting.

16. The method of claim 1 wherein the user's movement is corrected after receiving the alert.

17. The method of claim 18 wherein a session comprises a number of movements by the user or a distance gained by the user, the method further comprising the steps of:
alerting the user that the user's movement is away from the reference point by modifying the reference rhythm;
maintaining the modified rhythm until the movement is corrected; and
altering the rhythm back to the reference rhythm after correction of the user's movement; and
repeating the steps until the session is complete.

18. The method of claim 17 wherein a session comprises a distance of fifteen feet;
wherein the session is directed at correcting an incorrect gait; and
wherein the gait of the user is improved by about 1 to about 20 percent after one session.

19. The method of claim 1 wherein the reference point of movement is determined from a medical database comprising patients' medical records, patients' medical data, and/or patients' biofeedback data.

20. The method of claim 1 wherein the reference point of movement is determined from the user's physical state, textbooks, reference manuals, and/or the user's disability.

21. The method of claim 1 wherein the reference point of movement comprises the ultimate movement goal to be achieved or one of many movements necessary to achieve in order to achieve the ultimate movement goal.

22. The method of claim 4 wherein the rhythm is variable or fixed.

23. The method of claim 22 wherein the music and/or beat signals are supplemented with additional beat signals or music to emphasize specific parts of the pattern.

24. The method of claim 23 wherein the music and/or beat signals are supplemented with additional beat signals or music to transform the variable pattern to a fixed pattern.

25. The method of claim 24 wherein the rhythm of the user's movement is determined and the music and/or beat signals are modulated to synchronize with the user's movement.

26. The method of claim 1 further comprising providing spoken instructions in addition to the reference rhythm.

27. The method of claim 26 wherein the spoken instructions comprise movement instructions.

28. The method of claim 27 wherein the movement instructions comprise “heal-toe” in repetition, “up-down” in repetition, “left-right” in repetition, or “in-out” in repetition.

29. A method for evaluating speech of a user and providing biofeedback comprising:
setting a reference point for speech by the user, whereby the reference point corresponds to a reference rhythm;
providing the reference rhythm to the user to assist the user in maintaining the reference point;
sensing the user's speech;
comparing the user's speech to the reference point rhythm; and
alerting the user that the user's speech is away from the reference point by modifying the reference rhythm to a modified rhythm.

30. A system for evaluating movement of a user and providing biofeedback comprising:
a component for providing rhythm to the user;
a component for setting a reference rhythm;
one or more sensors for sensing movement of the user;
wherein the component for providing rhythm alters the reference rhythm upon incorrect movement of the user to alert the user to the incorrect movement; and
wherein altering the reference rhythm comprises modifying the reference rhythm to a modified rhythm.

31. The system of claim 30 wherein the reference rhythm is modified by increasing or decreasing the amplitude of the rhythm.

32. The system of claim 31 wherein the rhythm comprises a pattern of movements.

33. The system of claim 32 wherein the pattern of movements comprises music and/or beat signals.

34. The system of claim 33 wherein the music and beat signals are in synchronization.

35. The system of claim 33 wherein the amplitude is increased or decreased by increasing or decreasing the amplitude of the music, the beat signals or both.

36. The system of claim 35 wherein the amplitude is increased or decreased at specific parts of the pattern.

37. The system of claim 33 wherein the amplitude is increased by increasing the amplitude of the music, the beat signals or both.

38. The system of claim 33 wherein the amplitude is decreased by decreasing the music, the beat signals or both.

39. The system of claim 33 wherein the music comprises rock, pop, classical, jazz, hip hop, blues, alternative rock, rap or a combination thereof.

40. The system of claim 33 wherein the beat signal comprises sounds from a musical instrument or a metronome.

41. The system of claim 40 wherein the musical instrument comprises drums, symbols, a wind instrument, a string instrument, a piano, a harpsichord, an organ, or a combination thereof.

42. The system of claim 31 wherein the rhythm is further modified by increasing or decreasing the tempo, timbre, frequency, pitch, spectral content, and/or spatial location within the audio field of the full pattern or at various parts of the pattern.

43. The system of claim 42 wherein the user's movement is evaluated and the full pattern or parts of the pattern of the reference rhythm are modified to be in synchronization with the user's movement.

44. The system of claim 30 wherein the component for providing rhythm to the user alters the modified rhythm to the reference rhythm after the user has returned to the reference point movement.

45. The system of claim 44 wherein the reference rhythm is altered each time the user moves away from the reference point movement.

46. The system of claim 30 wherein the movement comprises walking, running, leg exercises, arm exercises, leg movements, arm movements, trunk movements, or weight lifting.

47. The system of claim 30 wherein the user's movement is corrected after receiving the alert.

48. The system of claim 30 wherein a session comprises a number of movement or a distance gained, wherein the system continues to alter the reference rhythm to a modified rhythm upon incorrect movement, and wherein the system continues to alter the modified rhythm back to the reference rhythm after movement is corrected, repeating the steps until the session is completed.

49. The system of claim 48 wherein a session comprises a distance of fifteen feet;
wherein the session is directed at correcting an incorrect gait; and
wherein the incorrect gait of the user is improved by about 1 to about 20 percent after one session.

50. The system of claim 30 further comprising a medical database comprising patients' medical records, patients' medical data, and/or patients' biofeedback for determining the reference point of movement for the user.

51. The system of claim 30 wherein the reference point of movement is determined from the user's physical state, textbooks, reference manuals, and/or the user's disability.

52. The system of claim 30 wherein the reference point of movement comprises the ultimate movement goal to be achieved or one of many movements necessary to achieve in order to achieve the ultimate movement goal.

53. The system of claim 30 wherein the rhythm is variable or fixed.

54. The system of claim 53 wherein the music and/or beat signals are supplemented with additional beat signals or music to emphasize specific parts of the pattern.

55. The system of claim 54 wherein the music and/or beat signals are supplemented with additional beat signals or music to transform the variable pattern to a fixed pattern.

56. The system of claim 55 wherein the rhythm of the user's movement is determined and the music and/or beat signals are modulated to synchronize with the user's movement.

57. The system of claim 30 further comprising a component for providing spoken instructions in addition to the reference rhythm.

58. The system of claim 57 wherein the spoken instructions comprise movement instructions.

59. The system of claim 58 wherein the movement instructions comprise “heal-toe” in repetition, “up-down” in repetition, “left-right” in repetition, or “in-out” in repetition.

60. The system of claim 30 further comprising one or more of:
an interface component linked to the component for providing rhythm;
a transceiver for receiving an transmitting signals;
an analog digital converter;
a storage device;
a digital signal processor;
a database;
wherein the transceiver is linked to one or more of the sensors, interface component, an analog digital converter;
wherein the analog digital converter is linked to one or more of the sensors and digital signal processor;
wherein the digital signal processor is linked to one or more of the analog digital converter, storage device and interface component;
wherein the storage device is linked to one or more of the interface component, and a digital signal processor; and
wherein the database is linked to one or more of the interface component, the transceiver and the sensors.
